(19) United States
(12) Patent Application Publication
(10) Pub. No.: US 2020/0143435 A1
(43) Pub. Date: May 7, 2020

(54) INFORMATION PROCESSING APPARATUS, INFORMATION PROCESSING SYSTEM, INFORMATION PROCESSING METHOD, AND PROGRAM

(71) Applicant: TOYOTA JIDOSHA KABUSHIKI KAISHA, Toyota-shi (JP)

(72) Inventors: Keiko SUZUKI, Tokyo (JP); Masatoshi TOKUDA, Kawasaki-shi (JP)

(73) Assignee: TOYOTA JIDOSHA KABUSHIKI KAISHA, Toyota-shi (JP)

(21) Appl. No.: 16/587,567

(22) Filed: Sep. 30, 2019

(30) Foreign Application Priority Data
Nov. 6, 2018 (JP) 2018-208666

(51) Int. Cl. G06Q 30/02; G06Q 50/30; G06Q 10/02; B60W 40/08; G06K 9/00; A61B 5/0402

(52) U.S. Cl. CPC G06Q 30/0282; G06Q 50/30; G06Q 10/02; B60W 2040/0872; G06K 9/00845; A61B 5/0402; B60W 40/08

(57) ABSTRACT

An information processing apparatus includes a first acquisition unit configured to acquire a value of a stress index measured for a user of a vehicle shared by a plurality of persons during use of the vehicle, a storage processing unit configured to store the value to be associated with the user and the vehicle into a storage unit, and a selection unit configured to select a vehicle recommended to the user based on the value stored to be associated with the user in the storage unit in response to a vehicle use request from the user.

### INCORPORATION BY REFERENCE

The disclosure of Japanese Patent Application No. 2018-208666 filed on Nov. 6, 2018 including the specification, drawings and abstract is incorporated herein by reference in its entirety.

### BACKGROUND

### 1. Technical Field

The present disclosure relates to an information processing apparatus, an information processing system, an information processing method, and a program.

### 2. Description of Related Art

In the related art, in a vehicle sharing service such as car sharing or a rental car, there is a technique of providing recommendation information to a user by using attribute data (a driving history or a rental history) of the user (for example, refer to Japanese Unexamined Patent Application Publication No. 2016-206715 (JP 2016-206715 A)). The recommendation information also includes indicating a recommended vehicle model or a vehicle model that cannot be recommended.

### SUMMARY

However, in JP 2016-206715 A, the recommendation information is not created from the viewpoint of a vehicle model comfortable to a user being recommended. Therefore, in JP 2016-206715 A, a recommended vehicle model cannot be said to be a vehicle model comfortable to a user.

The present disclosure has been made in light of the circumstances, and is directed to increasing a probability of recommending a vehicle that can be comfortably used by a user.

A first aspect of the present disclosure relates to an information processing apparatus including a first acquisition unit, a storage processing unit, and a selection unit. The first acquisition unit acquires a value of a stress index measured for a user of a vehicle shared by a plurality of persons during use of the vehicle. The storage processing unit stores the value to be associated with the user and the vehicle into a storage unit. The selection unit selects a vehicle recommended to the user based on the value stored to be associated with the user in the storage unit in response to a vehicle use request from the user.

Thus, it is possible to select a vehicle recommended to a user based on stress of the user for a vehicle.

Therefore, it is possible to increase a probability of recommending a vehicle that can be comfortably used by a user.

The information processing apparatus according to the first aspect may further includes a second acquisition unit configured to acquire evaluation information regarding the vehicle, input by the user, the storage processing unit may store the evaluation information to be associated with the user and the vehicle in the storage unit, and the selection unit may select a vehicle recommended to the user based on the value and the evaluation information stored to be associated with the user in the storage unit in response to the vehicle use request from the user.

Thus, it is possible to select a vehicle recommended to a user by taking into consideration not only stress for a vehicle but also a subjective evaluation of the user.

Therefore, it is possible to further increase a probability of recommending a vehicle that can be comfortably used by a user.

A second aspect of the present disclosure relates to an information processing system including an information processing terminal and an information processing apparatus configured to be connected to the information processing terminal via a network. The information processing terminal includes a calculation unit configured to calculate a value of a stress index based on biological information measured for a user of a vehicle shared by a plurality of persons during use of the vehicle. The information processing apparatus includes a first acquisition unit configured to acquire the value of the stress index, a storage processing unit configured to store the value to be associated with the user and the vehicle in a storage unit, and a selection unit configured to select a vehicle recommended to the user based on the value stored to be associated with the user in the storage unit in response to a vehicle use request from the user.

Thus, it is possible to select a vehicle recommended to a user based on stress of the user for a vehicle.

Therefore, it is possible to increase a probability of recommending a vehicle that can be comfortably used by a user.

A fourth aspect of the present disclosure relates to an information processing method of causing an information processing apparatus to execute: an acquisition procedure of acquiring a value of a stress index measured for a user of a vehicle shared by a plurality of persons during use of the vehicle; a storage procedure of storing the value to be associated with the user and the vehicle into a storage unit; and a selection procedure of selecting a vehicle recommended to the user based on the value stored to be associated with the user in the storage unit in response to a vehicle use request from the user.

Thus, it is possible to select a vehicle recommended to a user based on stress of the user for a vehicle.

Therefore, it is possible to increase a probability of recommending a vehicle that can be comfortably used by a user.

A fourth aspect of the present disclosure relates to a program causing an information processing apparatus to execute an acquisition procedure of acquiring a value of a stress index measured for a user of a vehicle shared by a plurality of persons during use of the vehicle; a storage procedure of storing the value to be associated with the user and the vehicle into a storage unit; and a selection procedure of selecting a vehicle recommended to the user based on the value stored to be associated with the user in the storage unit in response to a vehicle use request from the user.

Thus, it is possible to select a vehicle recommended to a user based on stress of the user for a vehicle.

Therefore, it is possible to increase a probability of recommending a vehicle that can be comfortably used by a user.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a diagram illustrating a system configuration example in an embodiment of the present disclosure;

FIG. 2 is a diagram illustrating a hardware configuration example of a server apparatus 10 in the embodiment of the present disclosure;

FIG. 3 is a diagram illustrating functional configuration examples of a user terminal 30 and the server apparatus 10 in the embodiment of the present disclosure;

FIG. 4 is a flowchart for describing examples of process procedures executed by the user terminal 30;

FIG. 5 is a flowchart for describing examples of process procedures executed by the server apparatus 10 in response to reception of use information;

FIG. 6 is a diagram illustrating a configuration example of a use history storage unit 14;

FIG. 7 is a diagram illustrating a configuration example of a priority storage unit 15;

FIG. 8 is a flowchart for describing examples of process procedures executed by the server apparatus 10 when a shared car 20 is reserved; and

FIG. 9 is a diagram illustrating an example of a vehicle model selection region in a reservation screen.

### DETAILED DESCRIPTION OF EMBODIMENTS

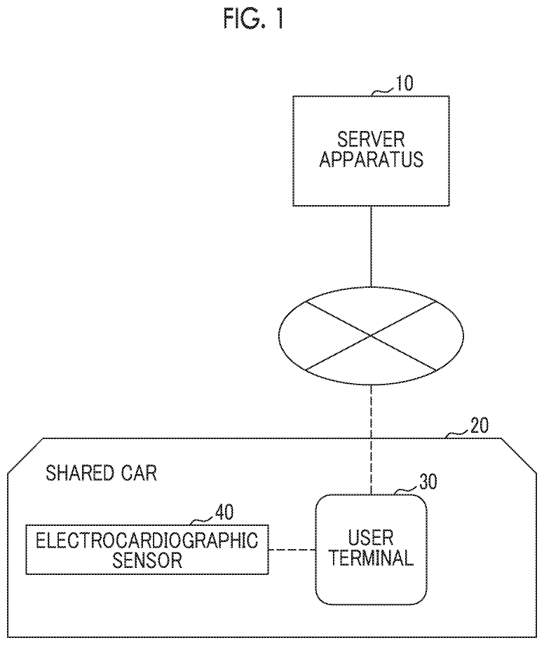

Hereinafter, with reference to the drawings, an embodiment of the present disclosure will be described. FIG. 1 is a diagram illustrating a system configuration example in an embodiment of the present disclosure. In FIG. 1, a server apparatus 10 is one or more computers that prepare a shared car 20 used in a car sharing service or a rental car service. The shared car 20 is a vehicle shared by a plurality of persons at different timings via, for example, a car sharing server or a rental car service.

A user terminal 30 is an information processing terminal owned by a user of the shared car 20. For example, a portable information processing terminal such as a smart phone or a tablet terminal may be used as the user terminal 30. Alternatively, an information processing terminal such as an on-vehicle apparatus mounted on the shared car 20 may be used as the user terminal 30. The user terminal 30 may perform communication with the server apparatus 10 via a predetermined communication network including a mobile communication network or the Internet having a plurality of base stations as ends. The user terminal 30 may perform communication with an electrocardiographic sensor 40 worn by a user (driver) of the shared car 20 during the use (riding or driving) of the shared car 20 via wireless communication. The electrocardiographic sensor 40 may employ any type of sensor as long as the sensor can measure an electrocardiogram of a user. However, the electrocardiographic sensor 40 preferably has a form of not hindering a driving operation of a user. For example, an electrocardiographic sensor having a garment shape, an electrocardiographic sensor having a wrist band shape, or an electrocardiographic sensor stuck to a part of a user's body may be used as the electrocardiographic sensor 40.

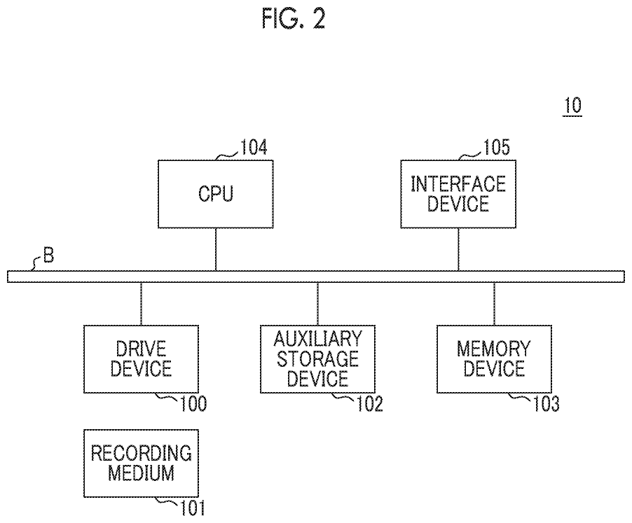

FIG. 2 is a diagram illustrating a hardware configuration example of the server apparatus 10 in the embodiment of the present disclosure. The server apparatus 10 in FIG. 2 includes a drive device 100, an auxiliary storage device 102, a memory device 103, a CPU 104, and an interface device 105 which are connected to each other via a bus B.

A program for realizing processes in the server apparatus 10 is provided by a recording medium 101 such as a CD-ROM. When the recording medium 101 recording the program is set in the drive device 100, the program is installed into the auxiliary storage device 102 from the recording medium 101 via the drive device 100. The program may be downloaded from another computer via a network instead of using the recording medium 101. The auxiliary storage device 102 stores the installed program, and also stores a needed file or data.

The memory device 103 reads the program from the auxiliary storage device 102, and stores the program thereinto, in a case where there is an instruction for starting the program. The CPU 104 realizes functions related to the server apparatus 10 according to the program stored in the memory device 103. The interface device 105 is used as an interface connected to a network.

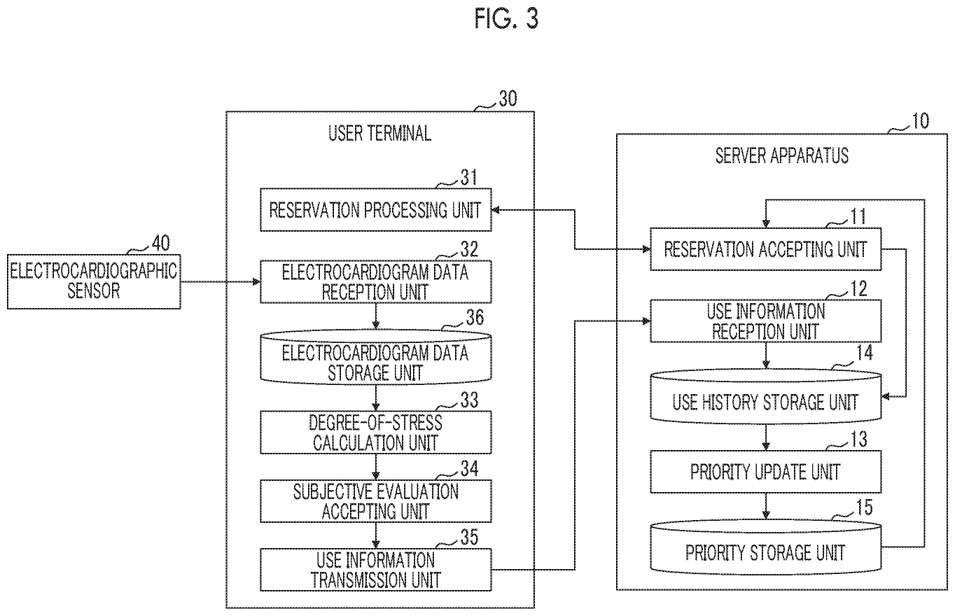

FIG. 3 is a diagram illustrating functional configuration examples of the user terminal 30 and the server apparatus 10 in the embodiment of the present disclosure. In FIG. 3, the user terminal 30 includes a reservation processing unit 31, an electrocardiogram data reception unit 32, a degree-of-stress calculation unit 33, a subjective evaluation accepting unit 34, and a use information transmission unit 35. The respective units are realized by processes that one or more programs (for example, applications) installed in the user terminal 30 cause a CPU of the user terminal 30 to execute. The user terminal 30 uses an electrocardiogram data storage unit 36. The electrocardiogram data storage unit 36 may be realized by using, for example, a memory or an auxiliary storage device of the user terminal 30.

The reservation processing unit 31 executes a process associated with a reservation for the use of the shared car 20. For example, the reservation processing unit 31 presents, to a user, selection candidates of the shared car 20 that is a reservation target, returned from the server apparatus 10 by transmitting a reservation start request to the server apparatus 10, and transmits information (hereinafter, referred to as “reservation information”) indicating a reservation of a selection candidate selected by the user, to the server apparatus 10.

The electrocardiogram data reception unit 32 sequentially receives electrocardiogram data transmitted from the electrocardiographic sensor 40 during the use of the shared car 20, and stores the received electrocardiogram data into the electrocardiogram data storage unit 36. Therefore, the electrocardiogram data during the use of the shared car 20 is stored in a time series in the electrocardiogram data storage unit 36. For example, short-range radio communication may be used as communication between the electrocardiographic sensor 40 and the user terminal 30.

The degree-of-stress calculation unit 33 calculates a value of a stress index based on the electrocardiogram data stored in the electrocardiogram data storage unit 36. The stress index is an index indicating the degree of stress height of a user. A well-known technique may be used to calculate a value of a stress index based on electrocardiogram data. For example, low frequency (LF)/high frequency (HF) may be used as a stress index. In the present embodiment, a value of a stress index will be referred to as the “degree of stress”. The degree of stress is an index value indicating that stress becomes higher as the value increases. In the present embodiment, it is considered that a user is in a more comfortable state as the degree of stress becomes lower.

The subjective evaluation accepting unit 34 accepts input of information (hereinafter, simply referred to as a “subjective evaluation”) indicating a subjective evaluation for the shared car 20, from the user of the shared car 20. The subjective evaluation is a value indicating the degree to which the user likes the shared car 20 used by the user. In the present embodiment, the subjective evaluation is expressed by values in five stages of 1 to 5. As a value becomes smaller, the subjective evaluation becomes higher (the degree to which the user likes the shared car 20 becomes higher).

For each use (from rental of the shared car 20 to return thereof) of the shared car 20, the use information transmission unit 35 transmits the user's identification information (hereinafter, referred to as a “user ID”) related to the use, reservation identification information (hereinafter, referred to as a “reservation ID”) related to the use, and information (hereinafter, referred to as “use information”) including the degree of stress and a subjective evaluation related to the use to the server apparatus 10.

On the other hand, the server apparatus 10 includes a reservation accepting unit 11, a use information reception unit 12, and a priority update unit 13. The respective units are realized by processes that one or more programs installed in the server apparatus 10 cause the CPU 104 to execute. The server apparatus 10 uses a use history storage unit 14 and a priority storage unit 15. Each storage unit may be realized by using, for example, the auxiliary storage device 102 or a storage device connectable to the server apparatus 10 via a network.

When a reservation start request transmitted from the reservation processing unit 31 of the user terminal 30 is received, the reservation accepting unit 11 selects the shared car 20 recommended to a user based on information stored in the priority storage unit 15 with respect to the user related to the reservation. The reservation accepting unit 11 transmits selection candidates of the shared car 20 that is a reservation target to the reservation processing unit 31 based on a selection result. The reservation accepting unit 11 receives reservation information transmitted from the reservation processing unit 31, and stores a record including the reservation information into the use history storage unit 14.

The use information reception unit 12 receives (acquires) use information transmitted from the use information transmission unit 35 of the user terminal 30, and stores the use information into a record storing reservation information corresponding to the use information from a recording group in the use history storage unit 14. Therefore, for each use of the shared car 20, the use history storage unit 14 stores use information (the degree of stress and a subjective evaluation) regarding the use.

In a case where new use information is stored in the use history storage unit 14, the priority update unit 13 calculates a priority regarding the user and the shared car 20 related to the use information based on the degree of stress and the subjective evaluation included in the use information, and updates a priority stored in the priority storage unit 15 with respect to the user and the shared car 20. The shared cars 20 are grouped on a vehicle model basis. In other words, a priority of each vehicle model is stored in the priority storage unit 15 for each user. The priority is an index used as a reference of a priority order when the shared car 20 that is a reservation target is recommended to the user. In the present embodiment, as a value of a priority becomes smaller, a priority order becomes higher.

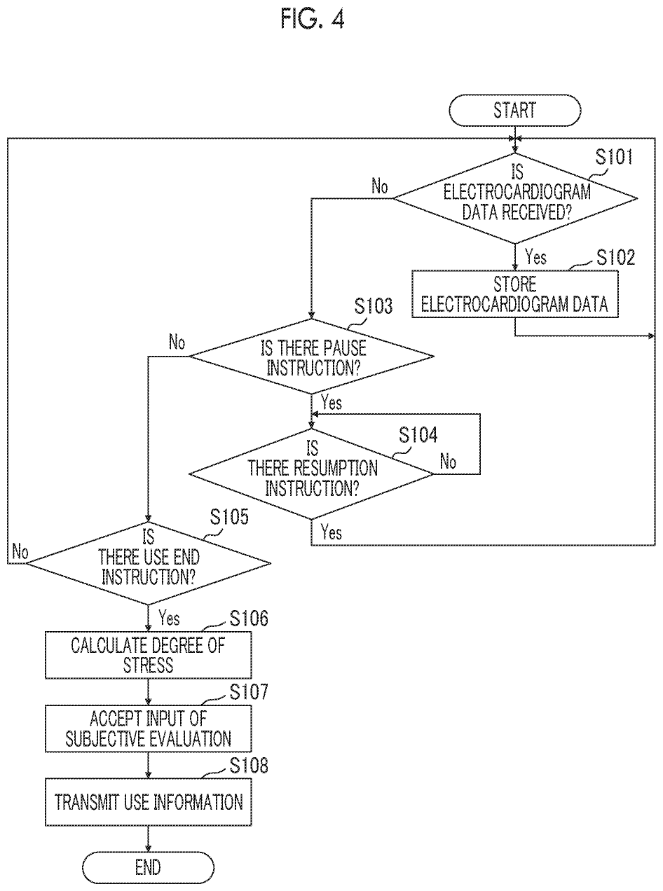

Hereinafter, a description will be made of process procedures executed by each of the user terminal 30 and the server apparatus 10. FIG. 4 is a flowchart for describing examples of process procedures executed by the user terminal 30. The process procedures in FIG. 4 are started in response to a predetermined operation performed on the user terminal 30 before a user gets on the shared car 20 and starts driving.

Whenever the electrocardiogram data reception unit 32 receives electrocardiogram data measured by the electrocardiographic sensor 40 from the electrocardiographic sensor 40 (Yes in S101), the electrocardiogram data reception unit 32 additionally stores the electrocardiogram data into the electrocardiogram data storage unit 36 (S102). Therefore, the electrocardiogram data is the electrocardiogram data storage unit 36 in a time series. Steps S101 and S102 are continuously executed until a pause instruction is input from the user (S103), or a use end instruction is input from the user (S105). The pause instruction is an instruction that is input to the user terminal 30, for example, in a case where the user temporarily stops the use (driving) of the shared car 20 due to getting-off. On the other hand, the use end instruction is an instruction that is input to the user terminal 30 when the user finishes the use of the shared car 20 (the user returns the shared car 20).

In a case where the pause instruction is input (Yes in S103), the electrocardiogram data reception unit 32 waits for execution of the process until a resumption instruction is input (S104). The resumption instruction is an instruction that is input to the user terminal 30 when the user the gets on the shared car 20 or resumes driving. When the resumption instruction is input (Yes in S104), step S101 and the subsequent steps are resumed.

On the other hand, when the use end instruction is input (Yes in S105), the degree-of-stress calculation unit 33 calculates the degree of stress based on a history of the electrocardiogram data stored in the electrocardiogram data storage unit 36 (S106). In other words, a single degree of stress is calculated with respect to one use. For example, LF/HF may be calculated as the degree of stress.

Subsequently, the subjective evaluation accepting unit 34 accepts input of a subjective evaluation from the user with respect to the shared car 20 used this time (S107). In other words, a single subjective evaluation is input with respect to one use. For example, the subjective evaluation accepting unit 34 may display a screen for selecting one from among subjective evaluations in five stages with 1 as the highest score and 5 as the lowest score on the user terminal 30, and may accept input of a subjective evaluation via the screen.

Subsequently, the use information transmission unit 35 transmits use information including a user ID related to the user terminal 30, a reservation ID regarding the present use of the shared car 20, the degree of stress calculated in step S106, and the subjective evaluation that is input in step S107, to the server apparatus 10 (S108). The user ID and the reservation ID may be input to the user terminal 30 by the user, for example, when the process procedures in FIG. 4 are started.

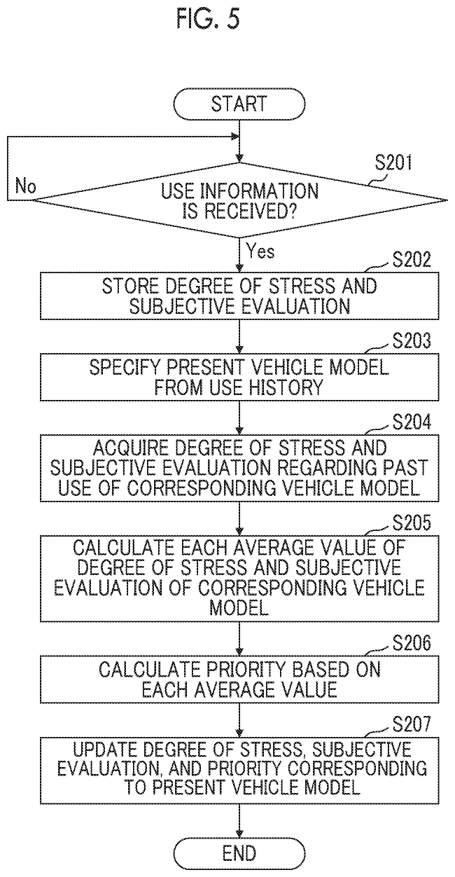

Next, process procedures executed by the server apparatus 10 will be described. FIG. 5 is a flowchart for describing examples of process procedures executed by the server apparatus 10 in response to reception of use information.

When use information (hereinafter, referred to as “target use information”) transmitted from any one of the user terminals 30 is received (acquired) (Yes in S201), the use information reception unit 12 stores the user ID, the reservation ID, the degree of stress, and the subjective evaluation included in the target use information into the use history storage unit 14 in association with each other (S202).

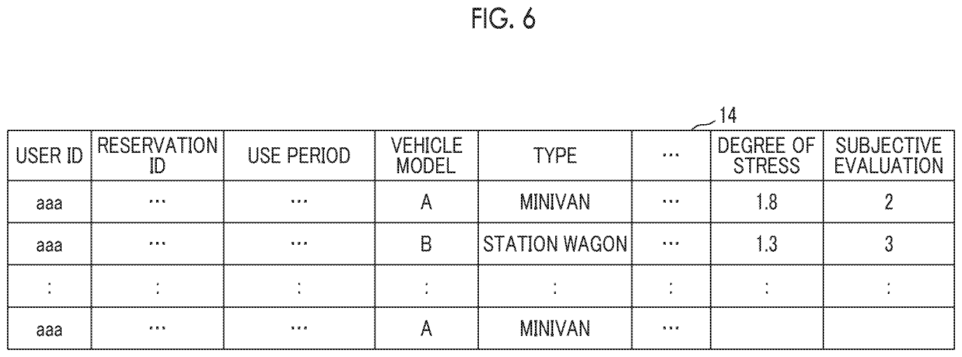

FIG. 6 is a diagram illustrating a configuration example of the use history storage unit 14. As illustrated in FIG. 6, the use history storage unit 14 is configured to include a user ID, a reservation ID, a use period, a vehicle model, a type, the degree of stress, and a subjective evaluation for each use (each reservation) of the shared car 20.

The user ID is a user ID of a user making a reservation for the shared car 20. The reservation ID is identification information of a reservation for the shared car 20. The use period is information indicating a start time and an end time of the use of the shared car 20. The vehicle model is a vehicle model of the shared car 20. The vehicle model is a product name of a vehicle used as the shared car 20. The type is information indicating a category to which the vehicle model of the shared car 20 belongs. In other words, the type is a concept of classifying a plurality of vehicle models. The degree of stress is the degree of stress for the shared car 20. The subjective evaluation is a subjective evaluation for the shared car 20.

As will be described later, among the items, values of items except the degree of stress and the subjective evaluation are stored into the use history storage unit 14 when the shared car 20 is reserved. Therefore, in step S202, the use information reception unit 12 retrieves a record including the user ID (hereinafter, referred to as a “target user ID”) and the reservation ID included in the target use information from the use history storage unit 14, and stores the degree of stress and the subjective evaluation included in the target use information into the corresponding record (hereinafter, referred to as a “target record”).

Subsequently, the priority update unit 13 specifies a vehicle model (hereinafter, referred to as a “target vehicle model”) of the shared car 20 related to the target use information by referring to the use history storage unit 14 (S203). The vehicle model may be specified by acquiring a vehicle of the vehicle model in the target record.

Subsequently, the priority update unit 13 acquires the degree of stress and a subjective evaluation stored in each record including the target user ID and the target vehicle model in the use history storage unit 14 (S204). In other words, the degree of stress and the subjective evaluation regarding the target user's each past use of an identical vehicle model are acquired. The acquired degree of stress and subjective evaluation also include the degree of stress and a subjective evaluation (that is, the degree of stress and a subjective evaluation regarding the present use) stored in the target record.

Subsequently, the priority update unit 13 calculates an average value of each of a degree-of-stress group and a subjective evaluation group acquired in step S204 (S205). In other words, the average value (hereinafter, referred to as a “degree-of-stress average”) of the degree-of-stress group and the average value (hereinafter, referred to as a “subjective evaluation average”) of the subjective evaluation group are calculated.

Subsequently, the priority update unit 13 calculates a priority based on the degree-of-stress average and the subjective evaluation average (S206). For example, the priority may be calculated by multiplying the degree-of-stress average by the subjective evaluation as follows. Priority=degree-of-stress average x subjective evaluation average

In this case, the subjective evaluation average functions as a weight for the degree-of-stress average.

Subsequently, the priority update unit 13 updates a content of the record related to the target user ID and the target vehicle model in the priority storage unit 15 (S207).

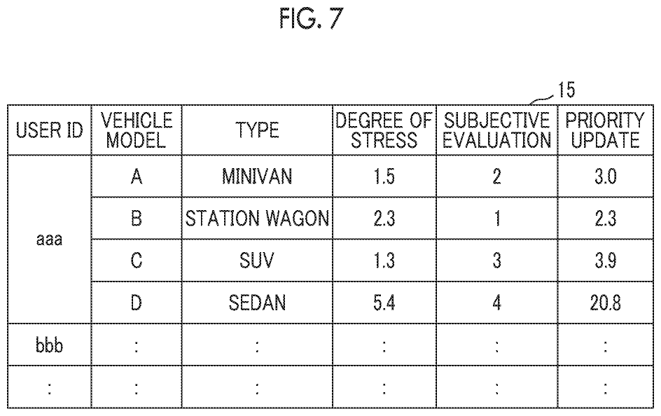

FIG. 7 is a diagram illustrating a configuration example of the priority storage unit 15. As illustrated in FIG. 7, the degree of stress, a subjective evaluation, and a priority are stored on a vehicle model basis for each user ID in the priority storage unit 15. In step S207, the degree of stress, the subjective evaluation, and the priority corresponding to the target user ID and the target vehicle model are updated. In other words, the degree of stress is overwritten by the degree-of-stress average. The degree of stress is overwritten by the subjective evaluation average. The priority is overwritten by the priority calculated in step S206.

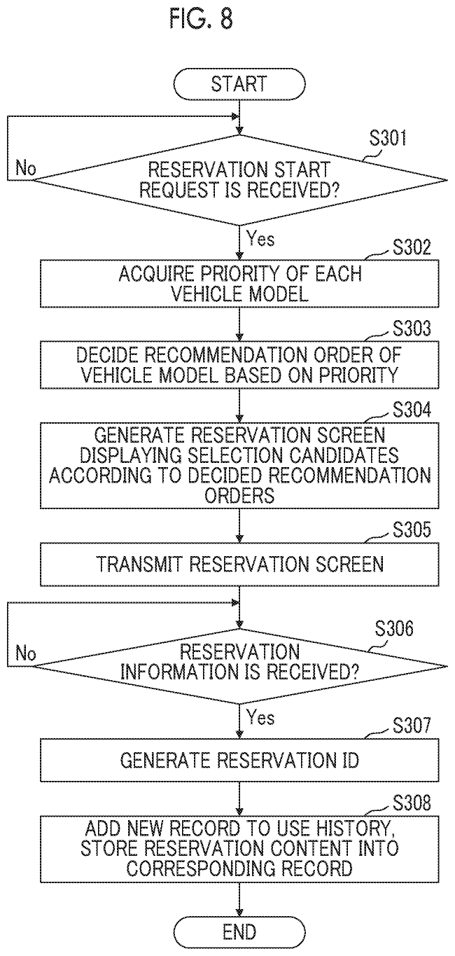

FIG. 8 is a flowchart for describing examples of process procedures executed by the server apparatus 10 when the shared car 20 is reserved.

When a reservation start request transmitted from any one user terminal 30 (hereinafter, referred to as a “target terminal”) is received (Yes in S301), the reservation accepting unit 11 executes step S302 and the subsequent steps. The reservation start request is a request transmitted by the user terminal 30 when a reservation instruction for the shared car 20 is input to the user terminal 30, and includes a user ID (hereinafter, referred to as a “target user ID”).

Subsequently, the reservation accepting unit 11 acquires a priority of each vehicle model associated with the target user ID from the priority storage unit 15 (FIG. 7) (S302). Subsequently, the reservation accepting unit 11 decides a recommendation order of a vehicle model based on the priority of each vehicle model (S303). Specifically, the reservation accepting unit 11 arranges (sorts) the vehicle models in an ascending order of the priority. Subsequently, the reservation accepting unit 11 generates display data for a screen (hereinafter, referred to as a “reservation screen”) including a region (hereinafter, referred to as a “vehicle model selection region”) in which the vehicle models are disposed as selection candidates according to the decided recommendation orders (S304). The display data may be generated, for example, in a Hyper Text Markup Language (HTML) format, and may be generated in other formats. Subsequently, the reservation accepting unit 11 transmits the display data for the reservation screen to the target terminal (S305). Subsequently, the reservation accepting unit 11 waits for reservation information to be received from the target terminal (S306).

When the display data for the reservation screen is received, the reservation processing unit 31 of the target terminal displays the reservation screen based on the display data.

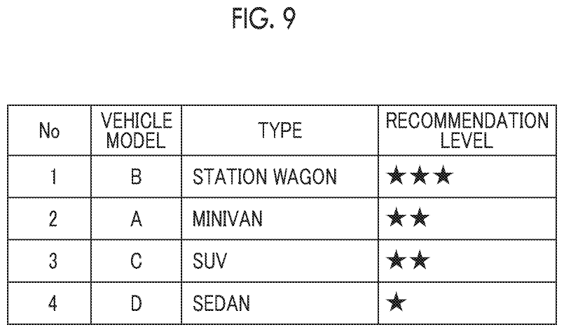

FIG. 9 is a diagram illustrating an example of a vehicle model selection region in the reservation screen. As illustrated in FIG. 9, vehicle models are arranged in an ascending order of the priority as use target selection candidates in the vehicle model selection region. Each vehicle model is assigned with a “recommendation level”. The “recommendation level” is information indicating the degree of recommendation with the number of asterisks. For example, the recommendation level may be determined by the reservation accepting unit 11 based on a threshold value group for dividing the priority into a plurality of stages. The order of arranging the vehicle models in FIG. 9 is based on the priorities of the respective vehicle models in which the user ID is “aaa” in FIG. 7.

When the user selects any one of the vehicle models in the vehicle model selection region, and inputs a reservation period or the like on the reservation screen, the reservation processing unit 31 of the target terminal transmits reservation information including the selected vehicle model and type or the input reservation period to the server apparatus 10.

When the reservation information is received (Yes in S306), the reservation accepting unit 11 of the server apparatus 10 generates the reservation information for a reservation ID (S307). Subsequently, the reservation accepting unit 11 generates a new record in the use history storage unit 14 (FIG. 6), and stores the target user ID and the reservation ID, and the reservation period, the vehicle model, and the type included in the reservation information into the record.

As described above, according to the present embodiment, a vehicle model recommended during a new reservation is selected (a recommendation order is decided) based on the degree of stress for each vehicle used by the user in the past. As a result, it is possible to recommend a vehicle giving relatively small stress to a user (in other words, a vehicle that the user feels relatively comfortable). Therefore, it is possible to increase a probability of recommending a vehicle that can be comfortably used by a user.

Since not only the degree of stress but also a subjective evaluation is considered, a subjective evaluation of a user can be reflected in recommendation of a shared car.

In the above description, a description has been made of an example in which a subjective evaluation is used as a weight, but the degree of stress may be used as a priority without being changed. In other words, a form in which a subjective evaluation is not considered may be employed. Alternatively, a subjective evaluation may be used as a priority without being changed.

In the above description, a description has been made of an example in which electrocardiogram data is used as biological information (vital information) of a user for calculating the degree of stress of the user, but other biological information such as a heart rate or a brain wave that is considered to be related to stress may be measured, and the degree of stress may be calculated based on the biological information. In this case, a sensor (a heart rate meter or an electroencephalograph) suitable for measurement target biological information may be mounted on a user.

In the above description, a description has been made of an example in which the user terminal 30 calculates the degree of stress, but the server apparatus 10 may calculate the degree of stress. In this case, the user terminal 30 does not include the degree-of-stress calculation unit 33, and the server apparatus 10 includes the degree-of-stress calculation unit 33. The use information transmission unit 35 of the user terminal 30 transmits use information including electrocardiogram data and a subjective evaluation stored in the electrocardiogram data storage unit 36, to the server apparatus 10. The degree-of-stress calculation unit 33 of the server apparatus 10 calculates a priority based on the electrocardiogram data included in the use information.

In the above description, a description has been made in which a priority of each vehicle model is calculated, and a recommendation order is used as an order of a vehicle model. In other words, the shared cars 20 are grouped by the vehicle models. However, the calculation unit of a priority is not limited to each vehicle model. For example, the calculation unit of a priority may be each type. Alternatively, a priority may be calculated for each group classified by a size of a vehicle or the presence or absence of various electric components (a navigation system or an electronic toll collection system (ETC)).

In the present embodiment, the server apparatus 10 is an example of an information processing apparatus. The use information reception unit 12 is an example of a first acquisition unit, a storage processing unit, or a second acquisition unit. The reservation accepting unit 11 is an example of a selection unit. The use history storage unit 14 is an example of a storage unit. The reservation start request is an example of a use request. The subjective evaluation is an example of evaluation information. The server apparatus 10 and the user terminal 30 are an example of an information processing system. The degree-of-stress calculation unit 33 is an example of a calculation unit.

As mentioned above, the embodiment of the present disclosure has been described in detail, but the present disclosure is not limited to the specific embodiment, and may be variously modified or changed within the scope of the spirit of the present disclosure disclosed in the claims.

What is claimed is:

1. An information processing apparatus comprising:
a first acquisition unit configured to acquire a value of a stress index measured for a user of a vehicle shared by a plurality of persons during use of the vehicle;
a storage processing unit configured to store the value to be associated with the user and the vehicle into a storage unit; and
a selection unit configured to select a vehicle recommended to the user based on the value stored to be associated with the user in the storage unit in response to a vehicle use request from the user.

2. The information processing apparatus according to claim 1, further comprising a second acquisition unit configured to acquire quire evaluation information regarding the vehicle, input by the user, wherein:
the storage processing unit stores the evaluation information to be associated with the user and the vehicle in the storage unit; and
the selection unit selects a vehicle recommended to the user based on the value and the evaluation information stored to be associated with the user in the storage unit in response to the vehicle use request from the user.

3. An information processing system comprising:
an information processing terminal; and
an information processing apparatus configured to be connected to the information processing terminal via a network, wherein:
the information processing terminal includes a calculation unit configured to calculate a value of a stress index based on biological information measured for a user of a vehicle shared by a plurality of persons during use of the vehicle, and
the information processing apparatus includes
a first acquisition unit configured to acquire the value of the stress index,
a storage processing unit configured to store the value to be associated with the user and the vehicle in a storage unit, and
a selection unit configured to select a vehicle recommended to the user based on the value stored to be associated with the user in the storage unit in response to a vehicle use request from the user.

4. An information processing method of causing an information processing apparatus to execute:
an acquisition procedure of acquiring a value of a stress index measured for a user of a vehicle shared by a plurality of persons during use of the vehicle;
a storage procedure of storing the value to be associated with the user and the vehicle into a storage unit; and
a selection procedure of selecting a vehicle recommended to the user based on the value stored to be associated with the user in the storage unit in response to a vehicle use request from the user.

5. A program causing an information processing apparatus to execute:
an acquisition procedure of acquiring a value of a stress index measured for a user of a vehicle shared by a plurality of persons during use of the vehicle;
a storage procedure of storing the value to be associated with the user and the vehicle into a storage unit; and
a selection procedure of selecting a vehicle recommended to the user based on the value stored to be associated with the user in the storage unit in response to a vehicle use request from the user.
